(19) United States
(12) Patent Application Publication
(10) Pub. No.: US 2002/0156320 A1
(43) Pub. Date: Oct. 24, 2002

(54) Enantioselective synthesis of valproic acid analogues

(76) Inventor: Heinz Nau, Hannover (DE)

Correspondence Address: MORGAN & FINNEGAN, L.L.P., 345 Park Avenue, New York, NY, 10154-0053, US

(21) Appl. No.: 10/175,009

(22) Filed: Jun. 19, 2002

Related U.S. Application Data
(63) Continuation of application No. 09/760,045, filed on Jan. 12, 2001, now abandoned.
(60) Provisional application No. 60/175,826, filed on Jan. 13, 2000.

(51) Int. Cl. C07C 51/353; C07C 231/18

(52) U.S. Cl. 562/505; 562/401; 562/606; 564/138

(57) ABSTRACT

This invention employs camphorsultam as a chiral recoverable auxiliary to provide a new method for manufacturing valproic acid and valproic acid amides that facilitates the enantioselective or diasteroselective production of valproic acid analogs on a larger scale.

This application claims priority to U.S. provisional application serial No. 60/175,826 filed Jan. 13, 2000 which is incorporated herein by reference in its entirety.

### FIELD OF THE INVENTION

This invention relates to a method for the production of enantiomerically or diastereomerically pure analogs of valproic acid and valproic acid amides.

### BACKGROUND OF THE INVENTION

As described in DE OS 42 31 085 A1, analogs of valproic acid according to the general formulas (Ia) and (Ib) are strong antiepileptics that only have a minor sedating and teratogenic effect:

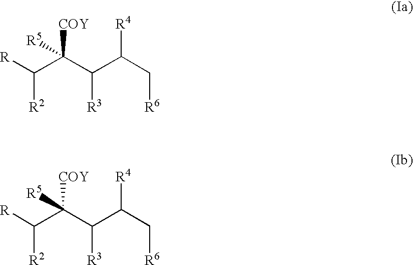

wherein R represents the group of

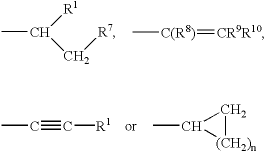

with n=1 to 6, Y is a hydroxy or amino group, and R¹, R², R³, R⁴, R⁵, R⁶, R⁷, R⁸, R⁹, and R¹⁰ each independently represent hydrogen or an unsaturated linear or branched C₁ to C₆ alkyl group.

Manufacturing these analogs, however, is both involved and costly. For example, they are synthesized from R-2-n-propyl-4-hexynoic acid using chiral oxazolidinone auxiliaries (Evans et al. J. Am. Chem. Soc. 1982, 104, pp. 1737-1739). Prior to saponification, the material must be treated using flash chromatography and HPLC. Most of all, separation using HPLC poses a big problem if the desired product is to be produced in any larger amounts. Despite any effort taken, crystalline oxazolidinone derivatives could not be produced so far, making it impossible to bypass the costly purification step by HPLC.

There is a need for a method for manufacturing said analogs of valproic acid and valproic acid amide that facilitates the enantioselective or diastereoselective production of valproic acid analogs at much larger quantities than prior art.

### BRIEF DESCRIPTION OF THE INVENTION

We apply the general method described below in which camphorsultam is used as a chiral auxiliary:

For producing compounds of the general formulas Ia and Ib,

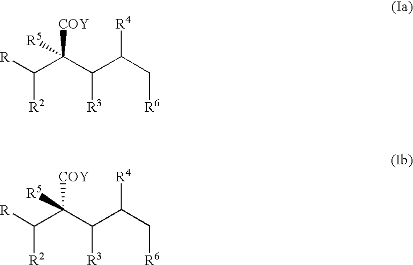

wherein R represents the group of

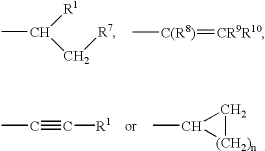

with n=1 to 6, Y is a hydroxy or amino group, and R¹, R², R³, R⁴, R⁵, R⁶, R⁷, R⁸, R⁹, and R¹⁰ each independently represent hydrogen or an unsaturated linear or branched C₁ to C₆ alkyl group,

a compound of the general formulas IIA ((+) camphorsultam) or IIb ((−) camphorsultam)

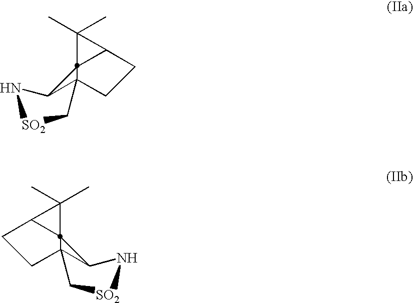

is deprotonated on nitrogen and subsequently acylated with a compound of the general formula III.

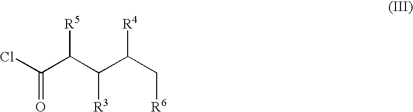

The resulting acyl compound is deprotonated in α position and subsequently alkylated using a compound of the general formula IV,

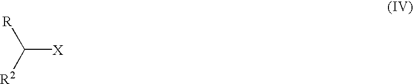

wherein X is a halogen.

Subsequent saponification yields the desired analog of valproic acid, the (+)-camphorsultam and (−)-camphorsultam yielding the R- or S-enantiomer, respectively.

### DETAILED DESCRIPTION OF THE INVENTION

Both antipodes of camphorsultam can be purchased. In addition, larger quantities of (+) camphorsultam or (−)-camphorsultam can be produced quickly and at a low price from camphorsulfonic acid in accordance with F. A. Davis, J. C. Towson, M. C. Weismiller, S. Lal and P. J. Carroll. J. Am. Chem. Soc. 1988, 110, pp. 8477-8482.

The general flow diagram below describes the enantioselective or diastereoselective synthesis of analogs of valproic acid and valproic acid amide using (+)-camphorsultam. Synthesis of analogs using (−)-camphorsultam takes place accordingly.

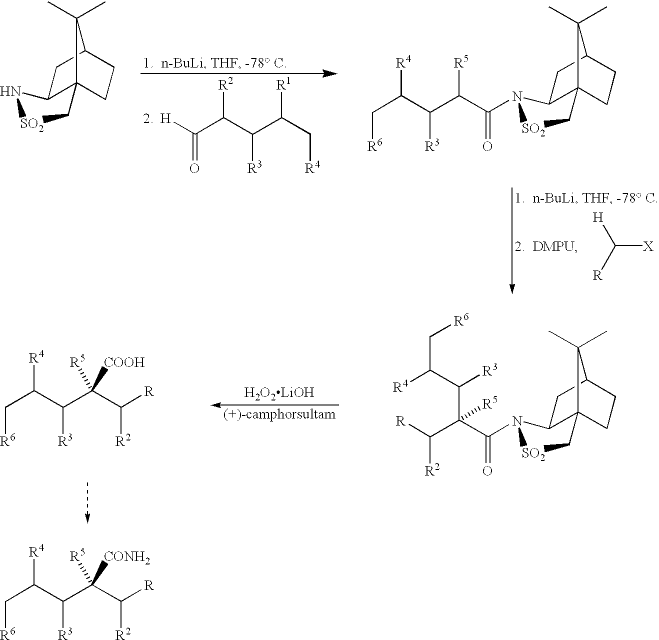

The process yields enantiomerically or diastereomerically pure derivatives of valproic acid whose carboxylate group can be converted into a carboxamide group. The respective carboxamides are produced without partial or complete racemization.

When using achiral substances, enantiomerically pure compounds whose enantiomeric excess (ee) is at 95% and above can be produced with this production method.

R-2-n-propyl-4-hexynoic acid, for example, is synthesized according to the following reaction scheme:

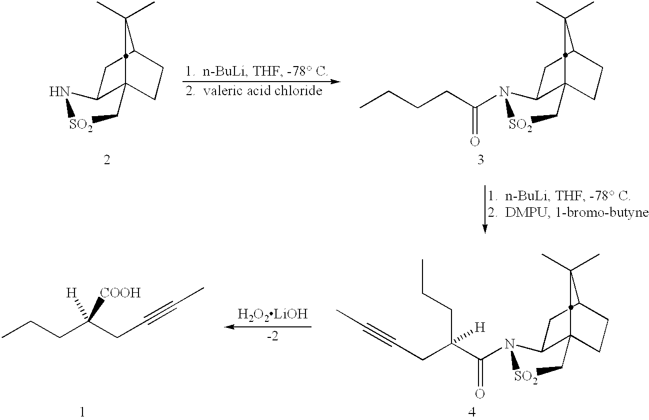

(+)-Camphorsultam (2) is deprotonated using n-BuLi and then reacted with valeric acid chloride. The resulting crystalline N-acylsultam (3) is purified, preferably by recrystallizing from hexane. The key step of the reaction is the stereoselective alkylation of the intermediary chiral enolate that is produced by deprotonating (3) using n-BuLi. It is required that this reaction is carried out in a polar aprotic solvent. The best yields are obtained when using highly carcinogenic HMPA. Alternatively, non carcinogenic 1, 3-dimethyltetrahydro-2(1H)-pyrimidinone (DMPU) can be used at a slightly lower yield of 70% after recrystallization. The crude N-acyl-sultam (4) already is highly stereoselective (de≈98% GC). Its diastereomeric purity can be increased to more than 99% (GC) by recrystallizing from methanol. The final product (1) is obtained in high yields by saponification using hydrogen peroxide and lithium hydroxide. The crude (+)-camphorsultam (2) that also accumulates is recovered by crystallization and purified by recrystallization. S-configured analogs such as S-2-n-propyl-4-decynoic acid can be obtained in a similar way using (−)-camphorsultam.

Use of camphorsultam according to the invention as a chiral auxiliary advantageously enables the synthesis of valproic acid analogs at a high efficiency and comparatively low expenditure. It is particularly advantageous that both enantiomers can be purchased or easily synthesized, which keeps the cost of the process relatively low.

Other advantageous embodiments of the method according to the invention are described in the subclaims.

The invention will be explained in more detail below by way of various examples.

### EXAMPLES

### General Synthesis of Acylated (+) or (−) camphorsultam

All reactions are carried out in purified and anhydrous solvents.

10.0 mmol of (+) or (−)-camphorsultam are dissolved in 20 ml of anhydrous THF and cooled down to −78° C. After adding 6.5 ml of n-BuLi (1.6M in hexane), the mixture is stirred at this temperature for 30 minutes. Subsequently, 10.3 mmol of acyl halide are added to this solution, and the mixture is kept agitated for another two hours while the solution heats up to room temperature. The mixture is stirred with water, extracted several times with ether, and the combined organic phases are dried. A crude product is obtained that can be purified using flash chromatography or by recrystallizing from hexane.

### General Method for the Stereoselective Alkylation of the N-acylsultam Obtained

A solution of 4.0 mmol of the purified N-acylsultam in 20 ml of anhydrous THF is cooled down to −78° C., and 2.5 ml of n-BuLi (1.6 M in hexane) are slowly added dropwise. The mixture is stirred for one hour at −78° C., then slowly intermixed with 4.5 mmol of alkyl halide dissolved in DMPU. The mixture is kept agitated for another 1.5 hours while it heats up to room temperature. A crude pro duct is obtained by mixing with water and extracting with ether. It can be purified by recrystallizing from methanol.

### General Method for Saponifying the Alkylated N-acylsultams obtained using Lithium Hydroxide and Hydrogen Peroxide

10.0 mmol of a 30% aqueous hydrogen peroxide solution mixed with 5.0 mmol of lithium hydroxide are added to a solution of 1.2 mmol of alkylated N-acylsultam in 10 ml of THF/water (4:1) at 0° C. The mixture is stirred for one hour at 0° C, then for 16 hours at 20° C., mixed with hydrochloric acid, and extracted with methylene chloride. The combined, organic phases are dried and evaporated to dryness. The residue is triturated in hexane to the insoluble (+)- or (−)-camphorsultam. The crude and soluble carboxylic acid can be purified using flash chromatography.

### Production of the Respective Analogs of the valproic acid amide

The conversion of the carboxylate group of valproic acid analogs into a carboxamide group is carried out using standard methods and will therefore not be described here.

### EXAMPLES

### Example 1

### Synthesis of R-2-n-propyl-4-hexynoic acid (1)

### Acylation of (+)-camphorsultam (2) with Valeric Acid Chloride

2.1 g (9.7 mmol) of (+) or (−)-camphorsultam (2) are dissolved in 15 ml of anhydrous THF and cooled down to −78° C. After adding 0.3 ml of n-BuLi (1.6 M in hexane) the mixture is stirred at this temperature for 30 minutes. Subsequently, 1.2 ml (10 mmol) of valeric acid chloride are added to this solution, and the mixture is kept agitated for another two hours while being allowed to warm to room temperatire. Aqueous workup and extraction with diethyl ether yield a crude product (3) (2.3 g, 7.8 mmol, 80%) that can be purified using flash chromatography or recrystallization from hexane.

### Stereoselective Alkylation of N-acylsultam (3)

A solution of 1.2 g (4.1 mmol) of recrystallized (3) in 20 ml of anhydrous THF is cooled down to −78° C., and 2.5 ml of n-BuLi (1.6 M in hexane) are slowly added by dropping. After stirring the mixture for one hour, a solution of 590 mg (4.5 mmol) of 1-bromo-2-butyne in 580 mg of DMPU is added. The mixture is kept agitated for another 15 hours while its temperature is increased to 20° C. Mixing with water and extraction with diethyl ether yields crude (4) (1.0 g, 3.4 mmol, 71%) that can be purified by recrystallizing from methanol.

¹H-NMR (CDCl₃): δ=0.9 (t,3H, ³J-7, 3 Hz), 0.98 (s, 3H), 1.21 (s, 3H), 1.28-1.58 (m, 5H), 1.73 (t, 3H, ⁴J-2.4 Hz), 1.75-1.93 (m, 4H), 2.09 (d, 2H, ³J-7.0 Hz), 2.44-2.47 (m, 2H), 3.14-3.19 (m, 1 H), 3.41-3.55 (m, 211) 3.9 (t, 1H, ³J-6.9 Hz); ¹³C-NMR (CDCl₃): δ=3.5 (p), 14.0 (p), 19.5 (p), 20.1 (s), 20.6 (p), 22.8 (s), 26.4 (s), 32.8 (2s), 38.4 (s), 44.6 (2t), 47.7 (g), 48.1 (q), 53.2 (s), 65.2 (t), 75.4 (q), 77.6 (q), 174.2 (q).

### Saponification of N-acylsultam (4) with Lithium Hydroxide and Hydrogen Peroxide

1.1 g (9.5 mmol) of 30% aqueous hydrogen peroxide and 200 mg (4.7 mmol) of lithium hydroxide are added to solution of 415 mg (1.2 mmol) of (4) in 10 ml of THF/water (4:1) at 0° C. The mixture is stirred for one hour at 0° C., then for 16 hours at 20° C., mixed with HCl, and extracted with methylene chloride. The dried extracts are evaporated. The residues are triturated in hexane to yield the insoluble (+)-camphorsultam (2) (yield: 90%). The crude R-2-n-propyl-4-hexinoic acid (1) (150 mg, 1.0 mmol, 83%) obtained can be purified by distillation or flash chromatography,

Boiling point: 94° C./0.2 mbar; ¹H-NMR (270 MHz/CDCl₃): δ=0.93 (t, 3H, ³J-7.3 Hz), 1.28-1.4 7 (m, 2H), 1.54-1.73 (m, 2 H), 1.76 (t, 3H, J-2.0 H z) 2.27-2.61 (m, 311), 11.45 (s(wide), 1H); ¹³C-NMR (270 MHz/CDCl₃): δ=3.3, 13.8, 20.2, 21.2, 33.2, 44.7, 75.8, 76.5, 181.6; m/z-211 (60%, M⁺-CH₃), 184 (24%, M⁺-C₃H₆), 183 (122%, M⁺-C₃H₇); [α]²⁵n =−2.2 (C 2.3, chloroform)

### Example 2

### Synthesis of S-4-methyl-2-n-propyl-4-pentenoic acid

### Acylation of (−)-camphorsultam, with valeric acid chloride

(−)-Camphorsultam is acylated in accordance with the directions given in Example 1.

### Stereoselective Alkylation of the Resulting N-acylsultam

A solution of 1.2 g (4.1 mmol) of the purified N-acyl-sultam in 20 ml of anhydrous THF is cooled down to −78° C., and 2.5 ml of n-BuLi (1.6 M in hexane) are slowly added by dropping. After stirring the mixture for one hour, a solution of 0.44 ml (4.5 mmol) of methallyl chloride in 600 mg of DMPU is added. The mixture is kept agitated for another 12 hours while its temperature is increased to 20° C. A crude product (yield 81%) is obtained by mixing with water and extracting with diethyl ether. It can be purified by recrystallizing from methanol.

### Saponification of the Alkylated N-acylsultam with Lithium Hydroxide and Hydrogen Peroxide

The last step of oxidative saponification using lithium hydroxide and hydrogen peroxide is carried out as described in Example 1. The resulting S-4-methyl-2-n-propyl-4-pentenoic acid is characterized by the following physical data:

Boiling point: 70-71° C./0.1 mbar; ¹H-NMR (270 MHz/CDCl₃): δ=0.88 (t, 3H, ³J-7.3 Hz), 1.20-1.65 (m, 4H), 1.70 (s, 3H), 2.07-2.20 and 2.28-2.40 (2m, 2H), 2.48-2.64 (m, 1H), 4.72 (d, 2H, ²J=11.0 Hz), 11.48 (s (wide), 1H); ¹³C-NMR (270 MHz/CDCl₃): δ=13, 9, 20, 5, 22, 2, 34, 1, 40, 3, 43, 7, 44, 5, 112, 2, 142, 6, 182, 6; m/z=213 (18%, M⁺-CH₃), 199 (4%, M⁺-C₂H₅), 186 (11%, M⁺-C₃H₆).

### Example 3

### Synthesis of S-3-methyl-2-(2-methylpentyl)-4-heptynoic acid

### Acylation of (−)-camphorsultam (4) with Methylheptanoyl Chloride

5.0 mmol of (−)-camphorsultam are dissolved in 10 ml of anhydrous THF and cooled down to −78° C. After adding 3.3 ml of n-BuLi (1.6 M in hexane), the mixture is stirred at this temperature for 30 minutes. Subsequently, a solution of 5.3 mmol of 4-methylheptanoyl chloride in 0.5 ml of anhydrous THF are added, and the mixture is kept agitated for another two hours while the solution heats up to room temperature. After aqueous processing and extraction with diethyl ether 3.6 mmol of crude product (yield: 72%) are obtained that can be purified by flash chromotography or recrystallizing from hexane.

4-Methylheptanoyl chloride is produced from the respective alcohol using known standard methods.

### Stereoselective Alkylation of the Resulting N-acylsultam

A solution of 3.6 mmol of the resulting and purified N-acylsultam in 15 ml of anhydrous THF is cooled down to −78° C. and slowly mixed with 2.2 ml of n-BuLi (1.6 M in hexane). The mixture is stirred for one hour at −78° C., then a solution of 4.2 mmol of 2-bromo-3-hexyne in 1.0 ml of DMPU is added by dropping. The mixture is kept agitated for another 15 hours while it is allowed to slowly heat up to room temperature. Mix with water and extract with diethyl ether. After processing 2.6 mmol (72%) of crude product are obtained that can be purified by recrystallizing from methanol.

2-bromo-3-hexyne can be produced, for example, from 3-hexyn-2-one using known methods.

The last step of oxidative saponification using lithium hydroxide and hydrogen peroxide is carried out as described in Example 1.

### Example 4

### Synthesis of R-2-cyclopropylmethyl-3,4-dimethyloctanoic acid

### Acylation of (−)-camphorsultam with 3 4-dimethyloctanoyl chloride

5.0 mmol of (−)-camphorsultam are dissolved in 10 ml of anhydrous THF and cooled down to −78° C. After adding 3.3 ml of n-BuLi (1.6 M in hexane), the mixture is stirred at this temperature for 30 minutes. Subsequently, a solution of 5.3 mmol of 3,4 dimethyloctanoyl chloride in 1.0 ml of anhydrous THF are added, and the mixture is kept agitated for another two hours at −78° C., then heated up to room temperature within another two hours. After aqueous processing and extraction with diethyl ether, 3.1 mmol of crude product (yield: 82%) are obtained that can be purified by flash chromatography or by recrystallizing from hexane.

3, 4-Dimethyloctanoy chloride is produced from the respective alcohol using known standard methods.

### Stereoselective Alkylation of the Resulting N-acylsultam

A solution of 3.1 mmol of the resulting purified N-acylsultam in 15 ml of anhydrous THF is cooled down to −78° C. and slowly mixed with 2.0 ml of n-BuLi (1.6 M in hexane). The mixture is stirred for one hour at −78° C., then a solution of 3.6 mmol of cyclopropylmethyl bromide (can be purchased at Acros Chimica) in 0.5 ml of DMPU is added by dropping. The mixture is kept agitated for another 15 hours while it is allowed to slowly heat up to room temperature. The mixture is combined with water and extracted with diethyl ether. Evaporation of the organic extracts yields 1.8 mmol (58%) of crude product that can be purified by recrystallizing from methanol.

The last step of oxidative saponification using lithium hydroxide and hydrogen peroxide is carried out as described in Example 1.

### Example 5

### Synthesis of R-2-(1-methylpropyl)-3-ethyl-4-methylhexanoic acid

### Acylation of (+)-camphorsultam (3) with Methylpentanoyl Chloride

3.2 mmol of (+)-camphorsultam are dissolved in 7 ml of anhydrous THF and cooled down to −78° C. After adding 2.0 ml of n-BuLi (1.6 M in hexane), the mixture is stirred at this temperature for 30 minutes. Subsequently, a solution of 3.3 mmol of 3-methylpentanoyl chloride in 1.0 ml of anhydrous THF are added, and the mixture is kept agitated for another two hours while the solution heats up to room temperature. After aqueous processing and extraction with diethyl ether, 2.6 mmol of crude product (yield: 82%) are obtained that can be purified by flash chromatography or by recrystallizing from hexane,

3-Methylpentanoyl chloride can be obtained from DL-3-methyl valeric acid using standard methods. DL-3-methyl valeric acid can be purchased at Acros Chimica.

### Stereoselective Alkylation of the Resulting N-acylsultam

A solution of 2.0 mmol of the resulting and purified N-acylsultam in 10 ml of anhydrous THF is cooled down to −78° C. and slowly mixed with 1.2 ml of n-BuLi (1.6 M in hexane). The mixture is stirred for one hour at −78° C., then a solution of 2.5 mmol of 3-iodo-4-methylhexane in 1.0 ml of DMPU is added by dropping. The mixture is kept agitated for another 15 hours while it is allowed to slowly heat up to room temperature. Mix with water and extract with diethyl ether. After processing, 1.4 mmol (70%) of crude product are obtained that can be purified by recrystallizing from methanol.

3-Iodo-4-methylhexane can be produced, for example, from 4-methylhexan-3-ol or hexane-3,4-diol using known methods.

The last step of oxidative saponification using lithium hydroxide and hydrogen peroxide is carried out as described in Example 1.

### Example 6

### Synthesis of R-2-(cyclopropylmethyl)-pentanoic acid

### Acylation of (+)-camphorsultam with valeric acid chloride

(+)-Camphorsultam is acylated with valeric acid chloride in accordance with the directions given in Example 1.

### Stereoselective Alkylation of the Resulting N-acylsultam

A solution of 1.2 g (4.1 mmol) of the purified N-acyl-sultam in 20 ml of anhydrous TEF is cooled down to −78° C., and 2.5 ml of n-BuLi (1.6 M in hexane) are slowly added by dropping. After stirring the mixture for one hour, a solution of 0.43 ml (4.5 mmol) of cyclopropylmethyl bromide in 600 mg of DMPU is added. The mixture is kept agitated for another 14 hours while its temperature is increased to 20° C. A crude product (yield 72%) is obtained by mixing with water and extracting with diethyl ether. It can be purified by recrystallizing from methanol.

### Saponification of the Alkylated N-acylsultam with Lithium Hydroxide and Hydrogen Peroxide

The last step of oxidative saponification using lithium hydroxide and hydrogen peroxide is carried out as described in Example 1. R-2-(cyclopropylmethyl)pentanoic acid is characterized by the following physical data.

Boiling point: 91-93° C./1.2 mbar; ¹H-NMR (270 MHz/CDCl ₃): δ=−0.08-0.12 (m, 2H), 0.28-0.48 (m, 2H), 0.61-0.76 (m, 1H), 0.89 (t, 3H, ³J=8.0 Hz), 1.21-1.68 (M, 6H), 2.46 (m, 1H), 12.04 (s (wide), 1H); ¹³C-NMR (270 MHz/CDCl₃): δ=4.3, 4.5, 9.0, 13.9, 20.5, 34.2, 37.2, 45.9, 183.5; m/z =213 (30%, M⁺-CH₃), 199 (89%, M⁺-C₂H₅), 186 (13%, M⁺-C₃H₆)

### Example 7

### Synthesis of R-2-(2-propynyl)octanoic acid

### Acylation of (+)-camphorsultam with octanoyl chloride

3.2 mmol of (+)-camphorsultam are dissolved in 8 ml of anhydrous THF and cooled down to −78° C. After adding 2.0 ml of n-BuLi (1.6 M in hexane), the mixture is stirred at this temperature for 30 minutes. Subsequently, a solution of 3.3 mmol of octanoyl chloride in 2.0 ml of anhydrous THF is added, and the mixture is kept agitated for another two hours while the solution warms to room temperature. After aqueous workup and extraction with diethyl ether, 2.6 mmol of crude product (yield: 82%) are obtained that can be purified by flash chromatography or by recrystallizing from hexane.

### Stereoselective Alkylation of the Resulting N-acylsultam

A solution of 2.0 mmol of the resulting purified N-acylsultam in 10 ml of anhydrous THF is cooled down to −78° C. and slowly mixed with 1.2 ml of n-BuLi (1.6 M in hexane). The mixture is stirred for one hour at −78° C., then a solution of 2.5 mmol of propargyl bromide in 1.0 ml of DMPU is added by dropping. The mixture is kept agitated for another 13 hours while it is allowed to slowly heat up to room temperature. Mix with water and extract with diethyl ether. After processing, 1.4 mmol (70%) of crude product are obtained that can do be purified by recrystallizing from methanol.

The last step of oxidative saponification using lithium hydroxide and hydrogen peroxide is carried out as described in Example 1. R-2-(2-propynyl)octanoic acid is characterized by the following physical data:

Boiling point: 82-84° C./0.1 mbar; ¹H-NMR (270 MHz/CDCl₃); δ=0.88 (t, 3H), 1.40 (m, 8H), 1.90 (m, 2H), 2.04 (t, 1H), 2.32-2.68 (m, 3H), 12.04 (s (wide), 1H).

We claim:

1. A method for enantioselective or diastereoselective manufacturing of compounds of the general formulas Ia and Ib, 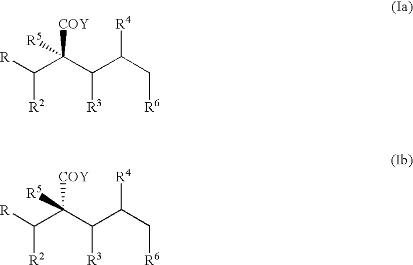
wherein R is selected from the group consisting of: 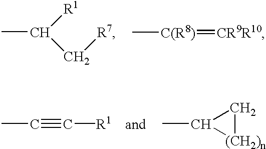
wherein n=1 to 6, Y is hydroxy or amino, and R¹, R², R³, R⁴, R⁵, R⁶, R⁷, R⁸, R⁹, and R¹⁰ each independently represents hydrogen or an unsaturated linear or branched C₁ to C₆ alkyl group;
wherein a compound of formula IIa ((+)-camphorsultam) or IIb ((−)-camphorsultam) 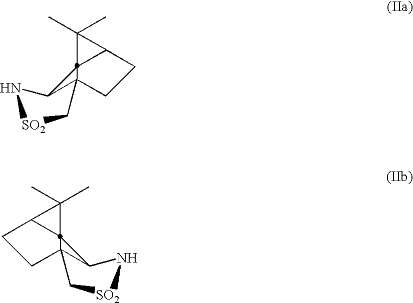
is deprotonated at nitrogen and subsequently acylated with a compound of the general formula III, 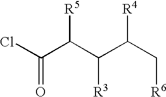
and the resulting acyl compound is deprotonated in ax position and subsequently alkylated with a compound of the general forumla IV, 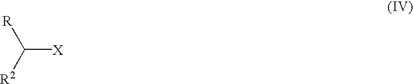
wherein x is a halogen atom;
and wherein the resulting alkylated N-acylsultam is saponified to cleave off the (+)-camphorsultam) (IIa), or (−)-camphorsultam (IIb), and the carboxylate group is optionally converted into a carboxamide group,

2. The method according to claim 1 further comprising the step of removing the undesired diastereomer of the alkylated N-acylsultam by fractional crystallization.

3. The method according to claim 1 wherein the N-acylsultam is deprotonated using a strong alkali metal base at low temperatures in a polar aprotic solvent.

4. The method according to claim 1 wherein the (+)-camphorsultam (IIa) or (−)-camphorsultam) (IIb)is recovered and reused after cleavage from said alkylated N-acylsultam.

5. The method according to claim 1 wherein the compound synthesized is selected from the group consisting of R-2-n-propyl-4-hexynoic acid, S-4-methyl-2-n-propyl-4-pentenoic acid, S-3-methyl-2-(2-methylpentyl)-4-heptynoic acid, R-2-cyclopropylmethyl-3,4-dimethyloctanoic acid, R-2-(1-methylpropyl)-3-ethyl-4-methylhexanoic acid, R-2-(cyclopropylmethyl)-pentanoic acid and R-2-(2-propynyl)octanoic acid.

6. The method according to claim 5 wherein the compound synthesized is R-2-n-propyl-4-hexynoic acid.
